(19) United States
(12) Patent Application Publication
(10) Pub. No.: US 2022/0101979 A1
(43) Pub. Date: Mar. 31, 2022

(54) Virtual Reality Therapy System and Methods of Making and Using Same

(71) Applicant: Viribus VR Labs, LLC, Oklahoma City, OK (US)

(72) Inventors: Brent Wheelbarger, Oklahoma City, OK (US); Robert Eskew, Oklahoma City, OK (US); Tony Matthews, Brookeville, MD (US)

(21) Appl. No.: 17/550,744

(22) Filed: Dec. 14, 2021

Related U.S. Application Data
(63) Continuation of application No. 17/252,120, filed on Dec. 14, 2020.
(60) Provisional application No. 62/685,706, filed on Jun. 15, 2018.

(51) Int. Cl. G16H 20/30; G06F 1/16; G06F 3/01

(52) U.S. Cl. CPC G16H 20/30; G06F 3/016; G06F 3/011; G06F 1/163

(57) ABSTRACT

A virtual reality therapy system is described. The virtual reality system includes a virtual reality headset, a garment, and an electronic device. The garment having at least one sensor, the at least one sensor configured to generate sensor data based upon movement. The electronic device is in communication with the virtual reality headset and receives the sensor data, or data based on the sensor data. The electronic device projects a virtual reality environment to the virtual reality headset based upon data generated by the at least one sensor of the garment.

### INCORPORATION BY REFERENCE OF RELATED APPLICATIONS

This application is a continuation of U.S. Ser. No. 17/25,120, filed Dec. 14, 2020, now abandoned, which is a 371 application of PCT/US19/37550, filed Jun. 17, 2019, which claims priority to the provisional patent application U.S. Ser. No. 62/685,706, filed on Jun. 15, 2018, the entire contents of which is expressly incorporated herein by reference.

### BACKGROUND

### 1. Field of Inventive Concept(s)

The presently disclosed and/or claimed inventive concept(s) relates, in general, to visual and/or touch feedback and virtual reality for patient therapy. More specifically, the presently disclosed and/or claimed inventive concept(s) relates to a virtual reality therapy system providing visual and/or haptic feedback to the user correlating to a virtual reality environment in which the therapy receiver is participating.

### 2. Background of Inventive Concept(s)

It is generally known that conventional rehabilitation programs are too short to ensure optimal results and do not motivate patients, in particular children, when they are not within the presence of their physical therapists. Studies have shown that the motivation of patients plays a critical role in treatment results. Researchers are finding that virtual environments, providing intriguing and competitive conditions, are one type of therapeutic tool that can increase the motivation of a patient and as well as their active participation in therapy. In turn, it has been found that the use of virtual reality (“VR”) environments lead to less therapy time being necessary for improving and/or regaining motor skills. There are many factors behind the use of VR in rehabilitation; VR can provide a variety of environments, including those like nature, designed to test performance and provide independent training. Such scenarios can be used to train and improve functional behavior in real life.

For example, but not by way of limitation, children with cerebral palsy (CP) can find even the simplest of tasks challenging—like standing still or maintaining good posture while seated. Good posture may be defined as posture presenting as skeletal alignment known as Ideal posture or Standard posture, that is the posture at which a line of gravity passes through the external auditory meatus, the shoulder joint, approximately midway from the trunk through the bodies of cervical and lumbar vertebrae, at the hip joint approximately through the greater trochanter of femur, anterior to the knee joint and anterior to the lateral malleolus. Traditional physical therapy techniques have been proven to help children with CP overcome such challenges. In particular, children with CP who adhere to a prescribed regimen of physical therapy generally avoid more significant and debilitating physical problems as adults—problems that often require invasive and expensive surgeries.

Physical therapists are often central to the team caring for persons with CP. They provide the patient with a structured exercise protocol while the patient is at the physical therapy facility. However, the ideal prescription for a patient with CP is structured exercises performed daily (i.e., movement every day has been found to be better than longer sessions a couple times per week). Patients are oftentimes unable to make it to physical therapy centers more than once per week, however. Reasons for such non-attendance include: (i) insurance often covers only weekly or bi-weekly sessions; (ii) patients may not be located near a therapist specializing in CP; (iii) generally, most physical therapy practitioners primarily focus on orthopedic issues and sports medicine; and (iv) many physical therapists don't work with pediatrics—thereby providing a scarcity of therapists for younger patients with CP. For these and other reasons, the inventors have found that only 10% of patients typically adhere to a good prescriptive regimen.

For these and other reasons, the inventors have found that an improved virtual reality therapy system involving visual and/or haptic feedback, prescribed physical therapies, and one or more virtual reality environments improves patient therapy outcomes—in particular, the therapy outcomes of children with CP.

### BRIEF DESCRIPTION OF THE SEVERAL VIEWS OF THE DRAWINGS

FIG. 1 is a rear-facing diagram of a user wearing an exemplary virtual reality therapy system according to the presently disclosed and/or claimed inventive concept(s).

FIG. 2 is a side-facing diagram of the user wearing the virtual reality therapy system according to the presently disclosed and/or claimed inventive concept(s).

FIG. 3 is an exemplary diagram of an electronic device of the virtual reality therapy system according to the presently disclosed and/or claimed inventive concept(s).

FIG. 4 is an exemplary diagram of a sensor of the virtual reality therapy system according to the presently disclosed and/or claimed inventive concept(s).

FIG. 5 is an exemplary process tree of a position collection process for the sensor of the virtual reality therapy system according to the presently disclosed and/or claimed inventive concept(s).

FIG. 6 is an exemplary process tree of an electronic device control process for an electronic device of the virtual reality therapy system according to the presently disclosed and/or claimed inventive concept(s).

FIG. 7 is an exemplary method of using the virtual reality therapy system according to the presently disclosed and/or claimed inventive concept(s).

FIG. 8 is a diagrammatic view of the user participating in a therapeutic environment in accordance with the presently disclosed and/or claimed inventive concept(s).

### DETAILED DESCRIPTION

Before explaining at least one embodiment of the present disclosure in detail by way of exemplary language and results, it is to be understood that the present disclosure is not limited in its application to the details of construction and the arrangement of the components set forth in the following description. The present disclosure is capable of other embodiments or of being practiced or carried out in various ways. As such, the language used herein is intended to be given the broadest possible scope and meaning; and the embodiments are meant to be exemplary—not exhaustive. Also, it is to be understood that the phraseology and terminology employed herein is for the purpose of description only and should not be regarded as limiting.

Unless otherwise defined herein, mechanical and technical terms used in connection with the present disclosure shall have the meanings that are commonly understood by those of ordinary skill in the art. Further, unless otherwise required by context, singular terms shall include pluralities and plural terms shall include the singular. The foregoing products and methods are generally produced and performed according to conventional methods well known in the art and as described in various general and more specific references that are cited and discussed throughout the present specification.

All patents, published patent applications, and non-patent publications mentioned in the specification are indicative of the level of skill of those skilled in the art to which this present disclosure pertains. All patents, and published patent applications referenced in any portion of this application are herein expressly incorporated by reference in their entirety to the same extent as if each individual patent or publication was specifically and individually indicated to be incorporated by reference.

All of the articles, systems, kits, and/or methods disclosed herein can be made and executed without undue experimentation in light of the present disclosure. While the articles, systems, and methods of the present disclosure have been described in terms of particular embodiments, it will be apparent to those of skill in the art that variations may be applied to the articles, systems, kits, and/or methods and in the steps or in the sequence of steps of the methods described herein without departing from the concept, spirit, and scope of the present disclosure. All such similar substitutions and modifications apparent to those skilled in the art are deemed to be within the spirit, scope, and concept of the present disclosure as defined by the appended claims.

As utilized in accordance with the present disclosure, the following terms, unless otherwise indicated, shall be understood to have the following meanings:

The use of the term “a” or “an” when used in conjunction with the term “comprising” in the claims and/or the specification may mean “one,” but it is also consistent with the meaning of “one or more,” “at least one,” and “one or more than one.” As such, the terms “a,” “an,” and “the” include plural referents unless the context clearly indicates otherwise.

The use of the term “at least one” will be understood to include one as well as any quantity more than one, including but not limited to, 2, 3, 4, 5, 10, 15, 20, 30, 40, 50, 100, etc. In addition, the use of the term “at least one of X, Y, and Z” will be understood to include X alone, Y alone, and Z alone, as well as any combination of X, Y, and Z. The use of ordinal number terminology (i.e., “first,” “second,” “third,” “fourth,” etc.) is solely for the purpose of differentiating between two or more items and is not meant to imply any sequence or order or importance to one item over another or any order of addition, for example.

The use of the term “or” in the claims or specification is used to mean an inclusive “and/or” unless explicitly indicated to refer to alternatives only or unless the alternatives are mutually exclusive. For example, a condition “A or B” is satisfied by any of the following: A is true (or present) and B is false (or not present), A is false (or not present) and B is true (or present), and both A and B are true (or present).

As used herein, any reference to “one embodiment,” “an embodiment,” “some embodiments,” “one example,” “for example,” or “an example” means that a particular element, feature, structure, or characteristic described in connection with the embodiment is included in at least one embodiment. The appearance of the phrase “in some embodiments” or “one example” in various places in the specification is not necessarily all referring to the same embodiment, for example. Further, all references to one or more embodiments or examples are to be construed as non-limiting to the claims.

As used in this specification and claim(s), the words “comprising” (and any form of comprising, such as “comprise” and “comprises”), “having” (and any form of having, such as “have” and “has”), “including” (and any form of including, such as “includes” and “include”), or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

As used herein, qualifiers like “substantially,” “about,” “approximately,” and combinations and variations thereof, are intended to include not only the exact amount or value that they qualify, but also some slight deviations therefrom, which may be due to computing tolerances, computing error, manufacturing tolerances, measurement error, wear and tear, stresses exerted on various parts, and combinations thereof, for example.

“Accessibility” refers to two separate attributes—accessibility by consumers and accessibility by developers.

“API” In computer programming, an application programming interface (API) is a set of routines, protocols, and tools for building software applications. An API expresses a software component in terms of its operations, inputs, outputs, and underlying types. An API defines functionalities that are independent of their respective implementations, which allows definitions and implementations to vary without compromising each other. In addition to accessing databases or computer hardware, such as hard disk drives or video cards, an API can ease the work of programming GUI components. For example, an API can facilitate integration of new features into existing applications (a so-called “plug-in API”). An API can also assist otherwise distinct applications with sharing data, which can help to integrate and enhance the functionalities of the applications. APIs often come in the form of a library that includes specifications for routines, data structures, object classes, and variables. In other cases, notably SOAP and REST services, an API is simply a specification of remote calls exposed to the API consumers. An API specification can take many forms, including an International Standard, such as POSIX, vendor documentation, such as the Microsoft Windows API, or the libraries of a programming language, e.g., Standard Template Library in C++ or Java API.

For the purposes of a virtual reality wearable technology, the term “ease of use” encompasses comfort and reliability.

A “module” in software is a part of a program. Programs are composed of one or more independently developed modules that are not combined until the program is linked. A single module can contain one or several routines or steps. A “module” in hardware, is a self-contained component.

A “software application” is a program or group of programs designed for end users. Application software can be divided into two general classes: systems software and applications software. Systems software consists of low-level programs that interact with the computer at a very basic level. This includes operating systems, compilers, and utilities for managing computer resources. In contrast, applications software (also called end-user programs) includes database programs, word processors, and spreadsheets. Figuratively speaking, applications software sits on top of systems software because it is unable to run without the operating system and system utilities.

A “software module” is a file that contains instructions. “Module” implies a single executable file that is only a part of the application, such as a DLL. When referring to an entire program, the terms “application” and “software program” are typically used. A software module is defined as a series of process steps stored in an electronic memory of an electronic device and executed by the processor of an electronic device such as a computer, tablet, smart phone, or other equivalent device known in the prior art.

A “software application module” is a program or group of programs designed for end users that contains one or more files that contains instructions to be executed by a computer or other equivalent device.

A “User” is any person using the computer system executing the method of the present invention. With regards to one or more embodiments of the present system, the term “user” is synonymous with “patient”.

A “therapist” is any person prescribing the use of the VR Therapy Environment or working as a main point of contact for the implementation of the VR Therapy Environment. A therapist may include an independent rehabilitation clinician such as a physical therapist, occupational therapist, speech-language pathologist, or any other medical professional.

The presently disclosed and/or claimed inventive concept(s) is a technological solution to the problems described above involving a lack of adherence to a good prescriptive regimen. In one embodiment, the virtual reality therapy device is especially adapted for use in therapeutic sessions with patients having cerebral palsy (“CP”). Generally, the virtual reality therapy device comprises a garment (e.g., vest, pants, jacket, body-suit, shoe, hat) connected to a smart phone (or other electronic device) application directing the use of a VR headset. The VR headset is, in one embodiment, a consumer-off-the-shelf (COTS) VR headset such as the OCCULUS RIFT® or OCCULUS GO® (Oculus VR, a division of Facebook Inc., Menlo Park, Calif.). The virtual reality therapy device enables therapist-directed VR game play incorporating visual and/or haptic physical responses to the user. The virtual reality therapy device may also be connected to a secure, HIPAA compliant, cloud-based website that collects data from the user as the user participates in the virtual game environment as directed by the therapist. The collection of data from the user allows: (1) physical therapists (and/or others involved in the patient's healthcare) to monitor and adjust the out-of-office exercise regimen; (2) physical therapists (and/or others involved in the patient's healthcare) to address patient care for patients who are geographically remote; (3) home caregivers to more efficiently maintain therapy regimens directed by the therapist; (4) patient's enjoyment of home programming through virtual game technology; and (5) patient's effectiveness during home programming by having more visual and physical interaction through VR. VR has a variety of potential benefits in therapeutic research and process and has shown encouraging results in healthcare and therapeutic processes (Chodos et al. 2010) to train people with disabilities who are using wheelchairs (Schultheis and Rizzo 2001) and in education (Caudell et al. 2003; Stansfield et al. 2005, Weiderhold 2006).

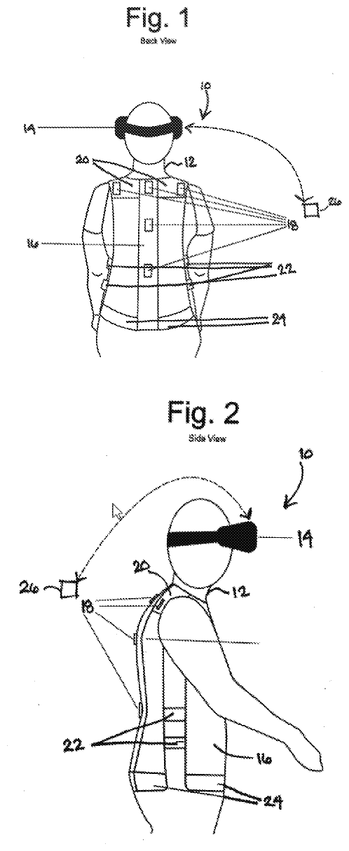

As shown in FIG. 1, the virtual reality therapy system 10 is generally shown as being worn by a user 12. The virtual reality therapy system 10 is a combined software and hardware platform for immersive virtual reality. In one embodiment, the virtual reality therapy system 10 comprises a virtual reality headset 14. The virtual reality headset 14 is a head-mounted device that provides a virtual reality or immersive experience to the user 12. Generally, virtual reality headsets or head-mounted displays (such as virtual reality headset 14) are devices capable of shutting the sensations from the real world, allowing the user (such as user 12) to concentrate on the virtual reality being provided. Virtual reality (VR) describes a human-computer interaction that is a multimedia interactive display in which the user (such as user 12) experiences a sense of presence or immersion in the virtual environment that changes in real time with head and body movements. Virtual reality is an advanced technology that enables humans to simulate, visualize, interact with, and manipulate existing places in the real world or, optionally, in imaginary worlds. The environment created by the virtual reality headset 14 can be video-based or completely computer-generated 3D graphics like a video game. In one embodiment, the virtual reality headset 14 is a consumer-off-the-shelf (“COTS”) VR headset such as the OCCULUS RIFT®, the OCCULUS GO®, GEAR VR® (Samsung Group, Seoul, South Korea), and/or HTC VIVE® (HTC Corporation, New Taipei City, Taiwan).

The virtual reality therapy system 10 also includes a garment 16. In the example shown in FIG. 1, the garment 16 is a vest, that is a piece of clothing that is sized to fit and extend over the abdomen, back, and chest of the user 12 but not on the arms or hands of the user 12. In one embodiment, the garment 16 is defined as an upper body garment (with or without arm components) embedded with sensors 18 and/or actuators, such as vibrating motors (not shown), allowing the garment 16 to track a user's movements and/or posture, translate the date indicative of the user's movements and/or posture into a virtual space, and, in certain embodiments, provide haptic feedback when the user performs a predetermined movement and/or comes into contact with a virtual object. The sensors 18 are generally an electronic device used to detect events or changes in the position or orientation of the user 12 (referred to herein as “positional data”) and transmit data about such changes to the smart phone and/or other electronic device 26 (described in more detail below). For example, the sensors 18 may form one or more inertial measurement system that may not depend upon external radio measurements. Instead, the inertial measurement system may keep track of position by measuring acceleration using accelerometers, rotation using gyroscopes, and magnetic fields using magnetometers, along one or more axis of the sensors 18. Movement along the one or more axis of the sensors 18 is called degrees of freedom. The inertial measurement system may be selected based on the degrees of freedom needed to provide precise positional data. In other embodiments, one or more of the sensors 18 may be a Global Positioning System (GPS) sensor. In one embodiment, at least one sensor 18 may be integrated into the virtual reality headset 14.

The number of sensors 18 on the garment 16 may be selected based on a required resolution of movement. The resolution of movement (resolution) is a measurement of the difference between the user's positional data and the user's “real-world” position. The user's real-world position is the user's body's actual position. By increasing the number of sensors 18, the electronic device 26 may be able to determine a more accurate representation of the user's position, that is, the user's position would be of a higher resolution. The higher resolution provides a therapist with more precise positional data thereby providing an opportunity to identify deficiencies in a user's movement and position more quickly, thereby increasing the efficiency of the VR Therapy. For example, if the number of sensors 18 along the spine of the user 12 is increased, the positional data from the sensors 18 may provide additional information on the curvature of the user's spine. The additional information may then be used by the therapist to adjust aspirational positional values and increasing the efficiency and efficacy of the VR Therapy Environment.

The location of the sensors 18 may be determined based on the skeletal, muscular, or other body location for which the therapist is prescribing treatment. For example, but not by way of limitation, if the therapist prescribed treatment for a shoulder impairment, the sensors may be placed at locations along the user's arm such as the front portion of the upper arm, the outside portion of the upper arm, the forearm, the shoulder blade, and the chest. By placing sensors along the arm in various locations, the therapist is not only able to determine whether the user 12 is able to raise their arm, but also determine whether, by raising their arm, the user is causing more damage elsewhere on their body, such as by straining other portions of their body to meet the aspirational positional value of the arm.

In another embodiment, the sensors 18 on garment 16 may be evenly distributed over the garment or the sensors 18 on garment 16 may be placed such that more sensors cover portions of the body that accommodate movement, for example, near joint locations such that a single axis joint may have two sensors, one on each member of the joint and a multi-axis joint may have sensors placed on each axis of each member of the joint. For a one-axis joint, with a sensor on both members of the joint, the electronic device 26 is able to account for common movements to determine the relative position of the two sensors. In one embodiment, at least one sensor 18 is placed on a substantially stationary portion of the user's body that is not near any joint, such as a chest sensor on the chest. By measuring the positional data of the chest sensor and a second sensor for a discrete period of time, the electronic device 26 can derive the resultant relative positions of the second sensor to the chest sensor, and thus, the position of the user's body on which the second sensor is positioned. Using simultaneous input from multiple sensors 18 allows the electronic device 26 to determine the user's posture and/or muscle alignment relative to the aspirational position.

The garment 16, in one embodiment, further includes connectors 20 that allow the garment 16 to be donned and removed by the user 12 in a convenient and efficient manner. The garment 16 may also include fasteners 22, in certain embodiments, that further allow the garment 16 to be donned and removed by the user 12 in a convenient and efficient manner. The connectors 20 and the fasteners 22 are, in one embodiment, contemplated as being made out of VELCRO® (Velcro Industries, United Kingdom) or any other type of hook and loop fastener. In general, hook and loop fasteners consist of two components: typically, two lineal fabric strips (or, alternatively, round “dots” or squares) which are attached (sewn or otherwise adhered) to the opposing surfaces to be fastened. The first component features tiny hooks; the second features even smaller and “hairier” loops. When the two components are pressed together, the hooks catch in the loops and the two pieces fasten or bind temporarily during the time that they are pressed together. In combination, the connectors 20 and the fasteners 22 allow for the garment 16 to be donned by the user and removed after use in a convenient and efficient manner. The connectors 20 and the fasteners 22 combine to provide the garment 16 with a configuration that can be adapted to the size and shape of the body of the user 12 so that the garment 16 is kept in close contact with the body of the user 12 and yet retains a desired ease of use for the user 12.

In one embodiment, the garment 16 further includes at least one weight 24 generally located near or adjacent to the bottom of the garment 16. The at least one weight 24 is attached to or otherwise incorporated within the garment 16 (e.g., positioned within a pocket) and provides a means for maintaining the garment 16 in close contact with the user 12. Maintaining close and intimate contact of the garment 16 with the user 12 assists with proper alignment of the sensors 18 to the actual placement of the body of the user 12 in both the real and VR environment. The at least one weight 24 can also prevent the hem or other aspect of the garment 16 from becoming out of close contact with the user 12 during unexpected movement of the user 12 and/or during unexpected weather conditions such as, for example but not by way of limitation, wind. In other embodiments, the garment 18 may be temporarily adhered to the user with a temporary body adhesive.

It should be understood, however, that the garment 16 can be in forms other than a vest. In other embodiments, the garment 16 may be a shirt, jacket, pants, socks, shoes, belt, or scarf. For example, the garment 16 can be in the form of any piece of clothing with sensors 18 capable of measuring positional data and worn by the user 12 such that the location of the sensors 18 are substantially stationary relative to user body locations next to which the sensor 18 is placed, that is, if the sensor 18 is on the garment 16 and the garment 16 is in the form of a vest, then while the user is performing the positional requirements of the prescribed game, the sensor 18, and thus that portion of the vest, should not move substantially from the placement of the vest or sensor 18 at the beginning of the game. In other embodiments, the garment 16 may be pants, or another leg covering. For example, if the therapist prescribes specific movements for the user's legs, the garment 16 may be pants. Likewise, if the therapist prescribes specific movements for the user's hands, the garment 16 may be a hand covering, such as gloves, if the therapist prescribes specific movements for the user's feet, the garment 16 may be a foot covering, such as socks or shoes, and if the therapist prescribes specific movements for the user's neck, the garment 16 may be a head covering, such as a hat or cap that only covers the top of the user's head or a balaclava that covers the majority of the user's head. Moreover, the garment 16 may be more than one piece of clothing such as pants and a jacket or a shirt, therefore enabling the therapist to prescribe games requiring more complex movement or positioning involving numerous body parts to target specific muscle groups.

In other embodiments, the sensors 18 may be attached directly to the user's body using a temporary body adhesive. Attaching the sensors 18 directly to the user's body allows precise placement of the sensors 18 to the location on the user's body the sensor is intended to measure. Additionally, the use of the temporary body adhesive prevents the sensors 18 from moving throughout a therapy session utilizing the VR therapy environment. One drawback of attaching sensor's directly to the user's body is a possible decrease in user comfort as well as minor variations in sensor placement between sessions thereby decreasing the consistency of the relative position of the sensors from one another.

In one embodiment, sensors are placed on the user 12 based on physical characteristics of the human body such as anatomic boney prominences. Anatomic boney prominences are simple anatomic locations that clinicians, the user 12, and other facilitators can consistently and reliably identify on the user's body. The use of anatomic boney prominences corresponds to posture and positioning of the user's body thereby providing precise measurements of positional data. Other facilitators may include any other person that may help place the sensors 18 on the user 12 or on the garment 16, that may help the user 12 don the garment 16, or that may otherwise be responsible for placing the sensors 18 in positions on or near the user 12 for accurate and precise positional data. Anatomic boney prominences may include, for example only and not by way of limitation, the distal acromion, C7 spinous process, the inferior angle of the scapula, which corresponds with the spinous process of the T7 vertebra, and the lumbar vertebra such as L3 and L4. Placing sensors at the previously specified anatomic boney prominences correlate to muscle activation of paraspinals, abdominal musculature, rhomboids, splenii group, and rotator cuff muscles groups as well as numerous secondary musculature. It is the activation and eventual strengthening of this musculature that the virtual reality therapy system 10 is directed—i.e., the sensors 18 communicate positional data of the user's body to the electronic device 26. Once positional data from the sensors 18 is within a threshold of the aspirational positional data, the positional data is indicative that the targeted musculatures have been activated and that strength training is being achieved.

When the user 12 is first prescribed the VR Therapy Environment, the sensors 18 should be calibrated to the user's body such that the electronic device 26 can reference the position of each sensor 18 on the user's body. Calibration may be done, for example, by informing the electronic device 26 the specific location of a specific sensor 18 on the user's body. The relative position of each remaining sensor 18 and thus the position (e.g., posture) of the user may then be determined based on the positional data of each other sensor 18 and the known relative position of each sensor 18 as placed on the garment 16. In other embodiments, the user 12 will first position themselves in a calibration position. When the user is in the calibration position, the positional data of each of the sensors 18 can be used as a reference against the known calibration position to determine the current position of each sensor 18, and thus, the current position (e.g., posture or muscle alignment) of the user 12.

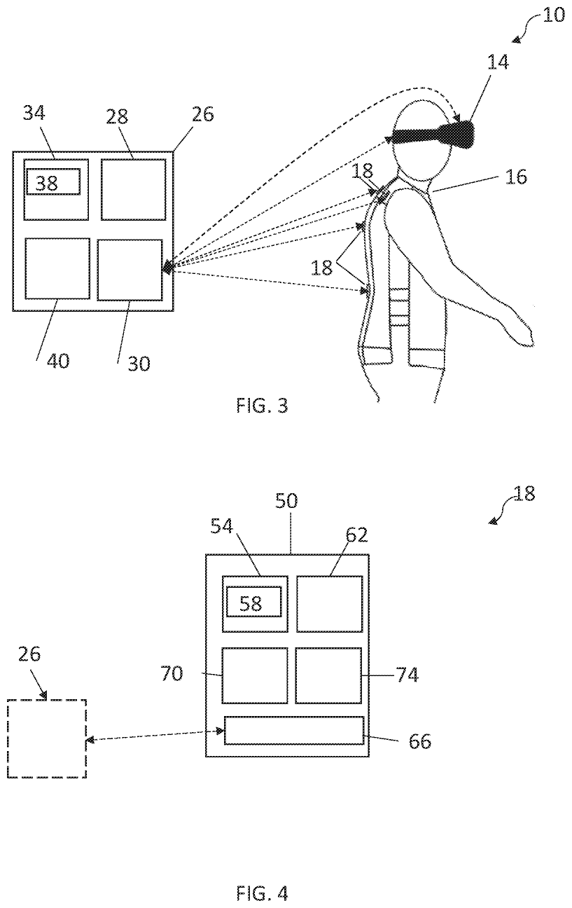

In the example shown in FIG. 3, the virtual reality therapy system 10 also includes the electronic device 26 (such as a smart phone or other device) comprising an application for connecting with the virtual reality headset 14 and the garment 16. The electronic device 26 contains a processor 28, at least one communication interface 30 providing electrical communication with the virtual reality headset 14 and the sensors 18 fixed to the garment 16, a user interface 40, and a non-volatile computer memory 34 (i.e., a non-transitory computer readable medium which may include random access memory, read only memory, flash memory, and/or the like. Such non-transitory computer readable mediums may be electrically based, magnetically based, optically based, and/or the like) storing computer instructions 38 that, when executed by the processor 28 monitor the positional data being generated by the sensors 18 and correlates such positional data with the VR environment created by the electronic device 26 and projected to the user 12 via the virtual reality headset 14. The processor 28 can either be a single processor or multiple processors working independently or together to execute the computer instructions. The user interface 40 provides the user 12 control of the electronic device 26 and may or may not be integrated into the electronic device 26. Additionally, the user interface 40 may or may not include an integrated sensor 18. In one embodiment, the user interface 40 allows the user 12 to take a snapshot or pause, start, stop, restart, power on, or power off the game, or any combination thereof. The user interface 40 may also provide the user 12 the ability to connect the sensors 18 to the electronic device 26 or to connect the virtual reality headset 14 to the electronic device 26. In one embodiment, the electronic device 26 is integrated into the garment 18 or the virtual reality headset 14. In one embodiment, the user interface 40 is provided for user interaction within the therapeutic environment. For example, and not by way of limitation, if the electronic device 26 were a smart phone, the virtual reality headset 14 may comprise the smart phone and a VR headset adapter wherein the smart phone provides both the screen on which the virtual reality environment is projected as well as the electronic device when placed within the VR headset adapter. One example of a VR headset adapter is Google Cardboard™ (Google LLC, Mountain View, Calif., USA).

The electronic device 26 is in electrical communication with the virtual reality headset 14 and the sensors 18. In one embodiment, the electrical communication between the electronic device 26 and at least one of the virtual reality headset 14 and the sensors 18 is wired. In an alternative embodiment, the electrical communication between the electronic device 26 and at least one of the virtual reality headset 14 and the sensors 18 is wireless. Wireless technologies compatible for use with the virtual reality therapy system include wireless technologies conforming to the requirements of BLUETOOTH® (Bluetooth Special Interest Group, Kirkland, Wash.), Wi-Fi, cellular data services such as GSM, CDMA, GPRS, W-CDMA, EDGE, LTE and/or 5G, low power wide area networks, wireless sensor networks, and/or mobile satellite communications, by way of example but not by way of limitation. In yet another embodiment, the electronic device 26 is in electrical communication with the virtual reality headset 14 utilizing a first communication scheme while the electronic device 26 is in electrical communication with the sensors 18 utilizing a second communication scheme, the first communication scheme and the second communication scheme being different wired constructions or being different wireless technologies.

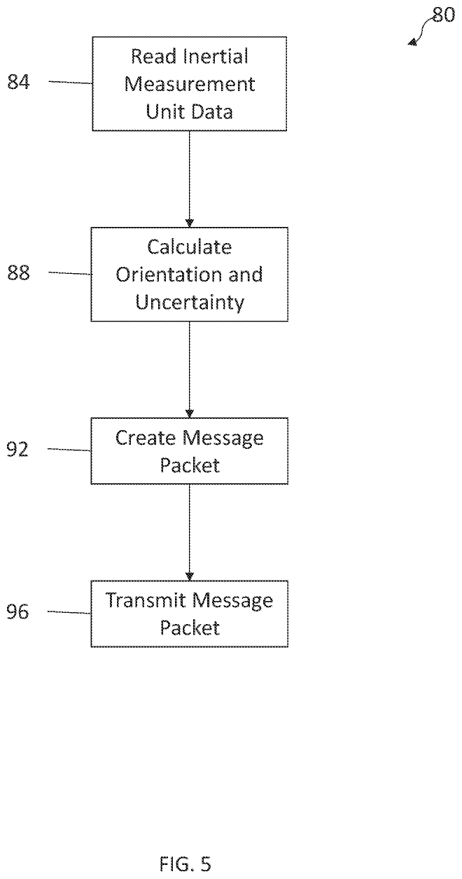

As shown in FIG. 4, in one embodiment, the sensor 18 is generally shown to comprise a controller 50 having a non-transitory memory 54 storing computer instructions 58, a sensor processor 62, a communication interface 66, a power system 70, and an inertial component 74. The communication interface 66 may be in electrical communication with the electronic device 26. The sensor processor 62 can either be a single processor or multiple processors working independently or together to execute the computer instructions 58. The sensor processor 62 may be selected such that power consumption is minimized. The power system 70 may be any type of battery, solar cell, or other portable power source suitable to be attached to the garment 16. The battery may be a rechargeable battery, a non-rechargeable battery, or other energy storage device. Battery chemistry of the battery compatible for use with the virtual reality therapy system include Nickel-cadmium, nickel-metal-hydride, lithium-ion, alkaline, lithium-ion-polymer, or lead acid, by way of example but not by way of limitation. The power system 70 may also be a wired power supply or a wireless power supply. The inertial component 74 may be any sensor designed to provide inertial data gathered by measuring inertia or one or more components of inertia, such as, by way of example only and not limited to, through the use of inertia sensors like gyroscopes, accelerometers, magnetometers, or any combination thereof.

As shown in FIG. 5, an exemplary embodiment of the position collection process 80 for collecting and sending positional data from the sensor 18 generally comprises the steps of collecting data from the inertial component 74 (step 84), calculating positional data of the sensor 18 and an orientation uncertainty (step 88), creating a message packet having the positional data (step 92), and transmitting the message packet via the communication interface 66 (step 96). The position collection process 80 may be encoded as computer instructions 58 stored in memory 54 and may be implemented when the sensor processor 62 executes the computer instructions 58.

The step 84 of collecting data from the inertial component 74 may be performed by the sensor processor 62 requesting sensor data from the inertial component 74 and storing the sensor data in the memory 54. In other embodiments, the inertial component 74 may send sensor data to the sensor processor 62 without a request from the sensor processor 62. The sensor data collected from the inertial component may be an aggregation of data from inertial sensors, may be data from all inertial sensors in the inertial component 74, or may be data from any combination of the inertial sensors in the inertial component 74.

The step 88 of calculating positional data of the sensor 18 and an orientation uncertainty may be performed by the sensor processor 62, by executing the computer instructions 58. Calculating positional data of the sensor 18 may involve performing mathematical operations on the sensor data or performing other operations on the sensor data in accordance with the computer instructions 58. Orientation uncertainty is a calculated value that mathematically and programmatically accounts for any measurement errors of the sensors.

The step 92 of creating a message packet having the positional data may be performed by the sensor processor 62. The message packet conforms to a data structure known to both the sensor 18 and the electronic device 26 to enable the electronic device 26 to process the message packet.

The step 96 of transmitting the message packet via the communication interface 66 may be performed by the sensor processor 62 sending the message packet to the communication interface 66. The communication interface 66 is complementary to the communication interface 30 and is a device capable of establishing electrical communication with the electronic device 26 via the communication interface 30.

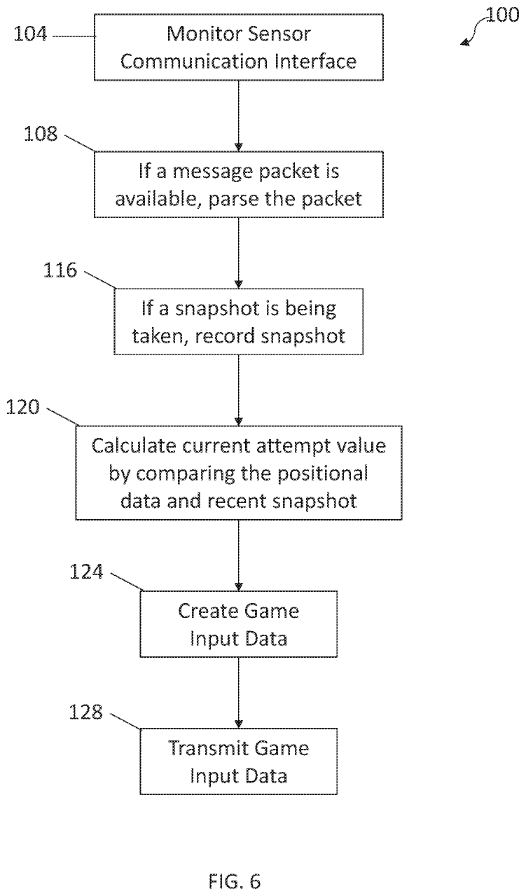

As shown in FIG. 6, an exemplary embodiment of the hub control process 100 for communicating with the sensors 18 and the virtual reality headset 14 generally comprises the steps of monitoring the communication interface 30 that is in electronic communication with the sensors 18 (step 104), if the message packet is available, parsing the message packet for the positional data (step 108), if a snapshot is being taken, recording the snapshot (step 116), comparing positional data to the most recent snapshot to determine a current attempt value (step 120), create game input data based at least in part on the current attempt value (step 124), and transmit game input data to the virtual reality headset 14 (step 128). The snapshot is the positional data at a first instance in time. The snapshot may then be compared to the positional data at a second instance in time in order to calculate the current attempt value (step 120), which may then be utilized to determine user improvement, that is, whether the positional data is closer to the “aspirational” position at a second instance in time than in a first instance in time, the aspirational position being preprogrammed preferred or “aspirational” positional data for the user 12 selected by the therapist. If the user 12 is improving, the game input data may be created to adjust the gameplay in a positive manner, that is adjusting the gameplay such that the user 12 is more easily able to complete in-game challenges, however, if the user 12 is not improving, the game input data may be created to adjust the gameplay in a negative manner, that is adjusting the gameplay such that the user 12 is more disadvantaged while trying to complete in-game challenges. For example, and not by way of limitation, if the game was centered around shooting a target, the positive manner may be increased accuracy or less drift while aiming, whereas the negative manner may be decreased accuracy or increased drift while aiming. The game input data is then transmitted to the virtual reality headset 14 to adjust the therapeutic environment (step 128).

The game input data may also be adjusted based on a relationship between the positional data, the snapshot, and the aspirational positional value, where the relationship may be defined as a mathematical function or combination of mathematical functions encoded in the computer instructions 38. As discussed in more detail below, when the user's positional data fails to meet or exceed the aspirational positional value, the game input data, and thus the VR therapy environment may be negatively impacted whereas when the user's positional data meets or exceeds the aspirational positional value, the game input data, and thus the VR therapy environment may be positively impacted.

For example, but not by limitation, if a first user, having a shoulder impairment, is prescribed a game wherein the first user was tasked with raising their arm and a second user, having a shoulder impairment, is prescribed the same game with the same task, the aspirational positional value may be a specified degree angle between the user's arm and body, the snapshot may be the initial value of the degree angle between the user's arm and body, and the positional data may be the current value of the degree angle between the user's arm and body. If the snapshot data is near the aspirational positional value for the first user but the snapshot data for the second user is not near the aspirational positional value for the second user, the electronic device 26 may alert the therapist to provide the therapist an opportunity to adjust the aspirational positional value for the first user in order to increase the treatment difficulty and may alert the therapist to provide the therapist an opportunity to adjust the aspiration positional value for the second user in order to make the aspirational positional value more reachable by the second user. Additionally, the electronic device 26 may adjust the gameplay such that a one degree difference in the angle of the first user's arm from the aspirational positional value has a greater negative impact on the first user's VR therapy environment than a more than one degree difference in the angle of the second user's arm from the aspirational positional value has on the second user's VR therapy environment. Thus, as the user 12 becomes more proficient in their movements and body placement, the electronic device 26 can encourage more exacting movements and body placement.

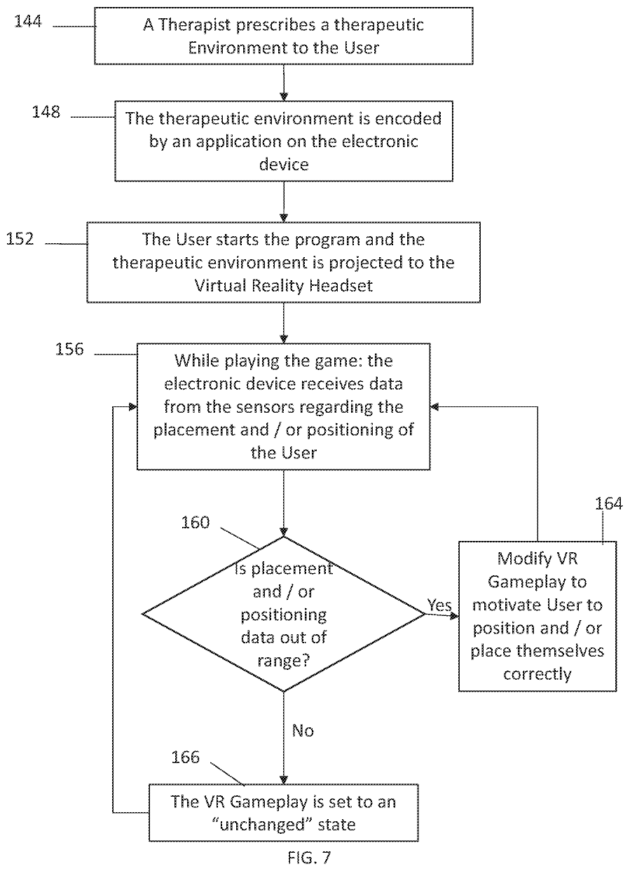

As shown in FIG. 7 depicting an exemplary method for using the virtual reality therapy system 140, in one embodiment, the therapist prescribes a therapeutic environment to the user 12 (step 144). The therapeutic environment is encoded by an application on the electronic device 26 (step 148) and is projected to the virtual reality headset 14 (step 152). The electronic device 26 receives data from the sensors 18 of the garment 16 regarding the placement and/or positioning of the user 12 (step 156). If the placement and/or positioning data is out of range, that is, the placement and/or positioning data is not within a threshold of the aspirational positional data (condition 160), such placement and/or positioning data is used by the electronic device 26 to provide positional feedback to the user 12 (step 164), otherwise the therapeutic environment projected to the virtual reality headset 14 is set to an “unchanged” state (step 168). The positional feedback provided to the user 12 may be, for example, adapting the therapeutic environment projected to the virtual reality headset 14, or providing haptic feedback, that is, the electronic device 26 may activate actuators that may be near any one or more sensor 18 wherein the haptic feedback may be scaled based in part on the difference of the positional data for a specific sensor and the aspirational data for that sensor. The threshold may be programmatically set by the electronic device 26 or may be set by the therapist. For example, the threshold may be adjusted such that of a user is within the threshold for an extended period of time, the electronic device 26 may decrease the threshold, thereby requiring more precise positioning or movement by the user.

For example, but not by way of limitation, the therapist prescribes a therapeutic environment comprising a space ship flying through a galaxy shooting aliens. As part of this therapeutic environment, the therapist has programmed the electronic device 26 (and/or selected a preprogrammed application existing on the electronic device 26) to receive positional data of the user 12 from the sensors 18. Such actual positional data of the user 12 is compared against aspirational positional data for the user 12 selected by the therapist. When the user 12 uses the virtual reality therapy system 10, the actual positional information of the user 12 is sent from the sensors 18 to the electronic device 26 and compared against the aspirational positional data set by the therapist. In the event that the actual positional data is substantially identical to and/or overlaps within an acceptable range of deviation from the aspirational positional data as set by the therapist, the therapeutic environment is unchanged and the user 12 can continue their VR gameplay. In the event that the actual positional data is not substantially identical to and/or overlaps within an acceptable range of deviation from the aspirational positional data as set by the therapist, the therapeutic environment is changed such that the VR gameplay is negatively impacted. For example, but not by way of limitation, when the actual positional information detected by the sensors 18 and sent to the electronic device 26 are determined to be “out of range”, the VR gameplay can be modified. For example, the VR gameplay can be slowed down. In another example, but not by way of limitation, when the actual positional information detected by the sensors 18 and sent to the electronic device 26 are determined to be “out of range”, the VR gameplay can be changed such that the ability to “shoot the aliens” is hampered in some manner—e.g., accuracy is impacted, the refresh rate of the shooting device is slowed, and/or the spacecraft itself is slowed in a perceptible manner.

As the VR gameplay is negatively impacted, the user 12 is motivated to position themselves once again such that their actual positional data sent by the sensors 18 is substantially identical and/or overlaps within an acceptable range of deviation from aspirational positional data as set by the therapist, so that the therapeutic environment is returned to an unchanged state and the user 12 can continue their VR gameplay unimpeded. In embodiments where the sensors 18 include vibrating motors, the sensors 18 can provide mechanical feedback to the user (in addition to the VR gameplay being impeded) about their actual positional data sent by the sensors 18 being substantially identical and/or overlapping within an acceptable range of deviation from the aspirational position as set by the therapist.

In other embodiments, if the user 12 maintains the aspirational position for a period of time longer than that set by the therapist or meets the aspirational movement a number of times in excess from that set by the therapist, the VR gameplay is positively impacted, that is the user 12 is provided with positive reinforcement by gaining more abilities within the game such as better weapons or better accuracy if playing a fighting game or more points per goal in a sports based game. In embodiments where the sensors 18 include vibrating motors, the sensors 18 can provide mechanical feedback to the user where the location of the sensor is near the aspirational position. This may be done in order to encourage the user to maintain the aspirational position.

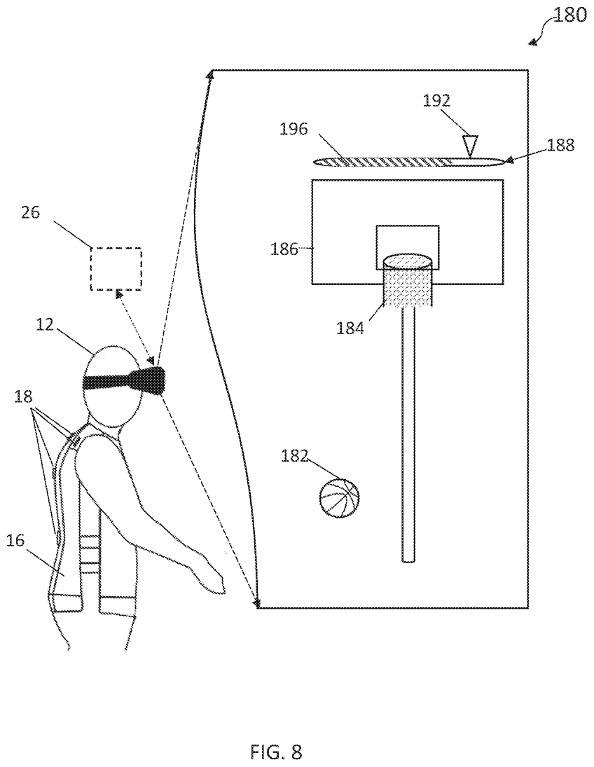

For example, but not by way of limitation, in one embodiment shown in FIG. 8, the therapeutic environment comprises a basketball game 180 wherein the user is tasked with competing against a computerized opponent in shooting a basketball 182 into hoop 184 of basketball goal 186. The user, by performing the tasks, creates muscle memory and strengthens postural control along the vertebrae. The basketball game is created by programming computer instructions 38 and loading the computer instructions 38 onto the electronic device 26 (and/or by selecting the basketball game from a preprogrammed list of games on the electronic device 26). The basketball game is programmed to receive positional data of the user 12 from the sensors 18 on garment 16. In order for the Basketball game to create muscle memory and strengthen postural control along the vertebrae of the user 12, the user 12 must don the garment 16 and hold as straight a posture as possible while a snapshot is taken, such as in step 116. The aspirational position is then determined based at least in part on the positional data of the snapshot at the first instance in time. The aspirational position may be set by the therapist or may be set by the electronic device 26. After the aspirational position is set, the basketball game requires the user 12 to position themselves at the aspirational position in order for the user 12 to shoot the basketball using the user interface 40. In some embodiments, the therapeutic environment is programmed so as to not require the use of any hand controllers to permit the user 12 to participate in the therapeutic environment. The electronic device 26 receives data from the sensors 18, such as in step 156 and provides the user 12 positional feedback based on a comparison of the user's current positional data and the user's aspirational data, such as in step 164. The positional feedback may be visual, that is, the positional feedback is determined by the electronic device 26 which displays the positional feedback on the virtual reality headset 14, the positional feedback may be haptic, that is, the positional feedback is the activation of actuators in the garment 16 wherein the actuators near the one or more sensors furthest from the aspirational position at that sensor 18 are activated by the electronic device 26, or the positional feedback may be any combination thereof. The visual positional feedback may be in the form of a positional gauge 188 having an aspirational indicator 192 on positional gauge 188 indicating the aspirational position and a positional indicator 196 on positional gauge 188 indicating the positional data in real-time. In other instances, the aspirational indicator 192 is a length of time and the positional indicator 196 indicates a period of time in which the user 12 has maintained the aspirational position. In either case, in some embodiments, in order for the user 12 to make a basket, the positional indicator must meet or surpass the aspirational indicator. Thus, in order to win the game, the user 12 must sit straight for long periods of time, thereby creating muscle memory and strengthening postural control along the vertebrae of the user 12.

For example, but not by way of limitation, another embodiment of the therapeutic environment prescribed by the therapist comprises a flying dragon game wherein the user 12 is tasked with flying a dragon along a path past obstacles from a first-person perspective by moving their body to direct the dragon. The user can shoot fire from the dragon by using the user interface 40 in order to eliminate certain obstacles. The user 12, by completing these tasks, strengthens and improves the range of motion along their spine, back, and core muscles. The user 12 starts at a neutral position and can move the dragon up by sitting up, down by bending forward, left by bending to the left, and right by bending to the right. In order for the user to pass a first obstacle, the user must position their body such that the positional data from the sensors 18 match or are within a range of aspirational data for the first obstacle thereby maneuvering the dragon around the first obstacle. If the user 12 is able to successfully maneuver the dragon around the first obstacle, the user will encounter a second obstacle. Again, the user must position their body such that the positional data from the sensors 18 match or are within a range of aspirational date for the second obstacle thereby maneuvering the dragon around the second obstacle. This pattern of the user positioning their body in order to successfully maneuver past obstacles may continue until all obstacles are successfully passed. The number of obstacles may be adjusted by the therapist in order to increase the number of times the user must position themselves to an aspirational position. By having the user 12 bend in different directions, this therapeutic environment requires that users move their back and shoulders to create constant motion thereby strengthening and improving range of motion along the spine, back, and core muscles.

The examples set forth above are not intended to be limiting. The skilled artisan will understand that other types of therapeutic environments can be created by following the teachings of the present disclosure.

Thus, in accordance with the present disclosure, there have been provided systems and devices, as well as methods of producing and using same, which fully satisfy the objectives and advantages set forth hereinabove—i.e., a virtual reality therapy system. Although the present disclosure has been described in conjunction with the specific drawings, experimentation, results, and language set forth hereinabove, it is evident that many alternatives, modifications, and variations will be apparent to those skilled in the art. Accordingly, it is intended to embrace all such alternatives, modifications, and variations that fall within the spirit and broad scope of the present disclosure.

What is claimed is:

1. A virtual reality therapy system, comprising:
a virtual reality headset;
a garment having at least one sensor associated therewith, the at least one sensor configured to generate sensor data based upon movement; and
an electronic device, in communication with the virtual reality headset and receiving the sensor data, or data based on the sensor data, wherein the electronic device projects a virtual reality environment to the virtual reality headset based at least in part upon data generated by the at least one sensor of the garment.

2. The virtual reality therapy system of claim 1, wherein the garment has at least one actuator associated therewith, the at least one actuator configured to generate movement.

3. The virtual reality therapy system of claim 1, wherein the garment is sized and dimensioned so as to fit about a back, abdomen, and chest of a user.

4. The virtual reality therapy system of claim 3, wherein the garment is a vest.

5. The virtual reality therapy system of claim 3, wherein the garment is a shirt.

6. The virtual reality therapy system of claim 3, wherein the garment is a belt.

7. The virtual reality therapy system of claim 3, wherein the garment is a jacket.

8. The virtual reality therapy system of claim 3, wherein the garment is sized and dimensioned so as to not extend upon any limb of the patient when the garment is being worn by the patient.

9. A method of using a virtual reality therapy system, comprising:
receiving a therapeutic virtual reality environment into an electronic device;
connecting the electronic device to a virtual reality headset and a garment, wherein the garment includes at least one sensor;
receiving by the electronic device positional data from the at least one sensor of the garment, the positional data being indicative of an actual position of the user while the user is wearing the garment;
comparing by the electronic device the actual position of the user to an aspirational position of the user; and
adjusting the therapeutic virtual reality environment at least in part in response to the comparison of the positional data to the aspirational position of the user by the electronic device.

10. The method of claim 9, wherein the aspirational position of the user is set by a therapist.

11. The method of claim 10, wherein the aspirational position is selected to create muscle memory of the user.

12. The method of claim 10, wherein the aspirational position is selected to strengthen postural control along vertebrae of the user.

13. The method of claim 10, wherein the aspirational position is selected to improve range of motion along the spine, back, and core muscles of the user.

14. The method of claim 9, wherein the electronic device is connected to the virtual reality headset by a wireless connection.

15. The method of claim 9, wherein the garment includes at least one actuator.

16. The method of claim 15 further comprising sending by the electronic device haptic data to the garment having the at least one actuator.

17. An electronic device comprising:
a processor; and
computer instructions stored on a non-transitory computer readable medium accessible by the processor, the computer instructions when executed by the processor cause the processor to:
receive positional data from at least one sensor positioned on a garment, and
project a virtual reality environment to a virtual reality headset based at least in part upon the positional data generated by the at least one sensor on the garment.

18. The electronic device of claim 17 further comprising computer instructions accessible by the processor, the computer instructions when executed by the processor cause the processor to send haptic feedback to the garment.

19. A non-transitory computer readable medium storing computer instructions that when executed by a processor cause the processor to:
receive a therapeutic virtual reality environment into an electronic device;
connect the electronic device to a virtual reality headset and a garment, wherein the garment includes at least one sensor;
receive by the electronic device positional data from the at least one sensor of the garment, the positional data being indicative of an actual position of the user while the user is wearing the garment;
compare by the electronic device the actual position of the user to an aspirational position of the user; and
adjust the therapeutic virtual reality environment at least in part in response to the comparison of the positional data to the aspirational position of the user by the electronic device.

20. The non-transitory computer readable medium storing computer instructions of claim 19, the computer instructions when executed by the processor cause the processor to send haptic feedback to the garment.

21. The non-transitory computer readable medium storing computer instructions of claim 19, wherein the user is receiving therapeutic treatment and the at least one sensor is positioned on the garment such that the positional data is sufficient for a therapist to determine efficacy of the therapeutic treatment.
